(19) United States
(12) Patent Application Publication
(10) Pub. No.: US 2020/0345535 A1
(43) Pub. Date: Nov. 5, 2020

(54) SNORE REDUCING MOUTHPIECE

(71) Applicant: Yuri Selukoff, San Francisco, CA (US)

(72) Inventor: Yuri Selukoff, San Francisco, CA (US)

(21) Appl. No.: 16/935,315

(22) Filed: Jul. 22, 2020

(51) Int. Cl. A61F 5/56

(52) U.S. Cl. CPC A61F 5/566

(57) ABSTRACT

A unitary body is placed inside the user's mouth, with an upper portion of a front face of the appliance and a lower portion of the front face of the appliance seated inside between the user's lips and the upper and lower front teeth and gums, where the teeth are loosely retained in teeth receiving channels in the body of the appliance.

### CROSS-REFERENCE TO RELATED APPLICATIONS

Not Applicable.

### STATEMENT REGARDING FEDERALLY SPONSORED RESEARCH OR DEVELOPMENT

Not applicable.

### SEQUENCE LISTING

Not applicable

### BACKGROUND OF THE INVENTION

The present invention is related to a mouthpiece that eliminates or reduces snoring.

### DESCRIPTION OF THE RELATED ART INCLUDING INFORMATION DISCLOSED UNDER 37 C.F.R. 1.97 and 1.98

Snoring is a common condition that can annoy other people who are near the snoring person and that may lead to sleep apnea.

Snoring is often caused by the relaxation of muscles in the mouth and throat, which causes restriction of the airway to the lungs. In sleep apnea, the airway is completely blocked temporarily, stopping breathing until the brain signals a resumption of breathing. Snoring and sleep apnea prevent deep restorative sleep. Both snoring and sleep apnea are potentially dangerous and may lead to long term health issues, although snoring is less likely to be dangerous than sleep apnea. The inventor has discovered that, when a person opens his or her mouth wide enough, the lower jaw pulls the connected muscles and other connected tissues in such a way that it forces the throat to open and the airway to the lungs to widen. When the mouth is opened at least as wide as a certain calculable angle, the corresponding widening of the airway causes reduction or elimination of snoring, but at the same time, the mouth should not be so far open that the sleeper feels that his or her mouth is being forced open. Therefore, if breathing though the mouth can be blocked while keeping the mouth open at the desired angle, the sleeper can breath comfortably though his or her nose and there will be no snoring. The user, however, must be able to regain instantly the ability to breath though the mouth if breathing through the nose becomes blocked during sleeping.

Many devices have been designed in an effort to reduce or eliminate snoring. Many grab the lower jaw by clamping the teeth and then force the jaw into a forward position, keeping the airway open even when the user is asleep and the jaw and throat muscles are relaxed. This approach works, but has significant disadvantages due to the fact that the lower jaw's being forced forward too much is not natural. These types of devices cause the teeth to move over time, just as a custom made dental retainer, but without any designed movement, thereby causing misaligned teeth and an unnatural change in the user's bite. Expensive dental intervention may be required in order to correct the user's bite. Further, temporomandibular joint (TMJ) disorder is a common side effect of using such devices. TMJ disorder may cause severe pain in the jaw joints, headaches, noises from the jaw joints, like cracking or popping, locking of the jaw, so that it is hard to open or close the jaw, ringing in the ears, muscle spasms in the jaw, swelling of the jaw or sensitivity of one or more teeth.

A few examples of patented designs include, for example, Samelson, U.S. Pat. No. 4,169,473 A1, discloses an Anti-Snoring and Anti-Bruxism Device. The device 10 comprises a molded one-piece body with an upper U-shaped trough 18 that receives the upper teeth 58 and a corresponding lower U-shaped trough 20 that receives the lower teeth 60 and the user's tongue 54 slides into the tongue receiving socket 14. This device appears to block the user's airway completely in all cases, forcing the user to breath only though his nose, which could be dangerous. Further, Samelson '473 keeps the user's tongue in a forward position by having the tongue inserted into a pliable cavity that uses a vacuum created by the insertion of the tongue into the cavity. This tongue position seems unlikely to be retained throughout the night and does not reduce snoring in many cases.

Hays, U.S. Pat. No. 5,277,202 A1, discloses an Easy Fit Anti-Snoring Device comprising a semi-circular arch having a forward wall 18 and a truncated rear wall 20, forming a teeth receiving trench or channel 32. (Column 4, lines 4-8). The lower teeth engage the ramp structure 24, which is the lower part of the device 10. In the center of the ramp structure 24 is an opening or air passage 28. Hays '202 has a large rectangular opening in the middle of its face, so that mouth breathing is always continuing, even when the user breathes through his nose, as the user's mouth is not ever wholly blocked.

Snyder, U.S. Pat. No. 5,462,066 A1, discloses a Snoring Relief Device comprising a custom molded mouthpiece with the vertical walls of the mouthpiece becoming shorter toward the rear of the mouth. The device 10 includes a exterior surface 16 that appears to cover the mouth and lips. The device 10 includes a formed trough or channel for retaining the upper teeth (FIG. 6) and a lower channel for retaining the lower teeth (FIG. 7). The entire appliance fits inside the user's mouth, as the appliance permits “the user's lips to achieve a closed condition.” (Column 6, lines 38-39) An upper U-shaped channel retains the upper teeth and a corresponding lower U-shaped channel retains the lower teeth in this custom fitted hot-molded appliance.

KR101828841 from Korea, filed Nove. 11, 2016, discloses a Snoring Prevention Device comprising an appliance that fits mostly inside the user's mouth, having an upper shelf 112 that the upper teeth rest on and a lower shelf 114 that the lower teeth rest on. Three air flow holes 122, 122, 124 are formed through the appliance and are tapered as they pass from the front to the rear (See FIGS. 4, 5). The upper port of the front of the main body 100 contacts the user's inner surface of the upper lip. This device also allows for mouth breathing at all times.

KR20060089860 from Korea, filed Feb. 4, 2005, discloses a Prevention Device for Snoring and Bruising (Prevention Device for Snore and Bruxism) comprising that resembles a standard mouthpiece, such as is used in many sports, but with a vent opening 21 in the center. The outer departure prevention portion 20 retains the appliance. This appliance pulls the lower jaw forward, resulting in the disadvantages discussed above.

Pulling the jaw forward creates an unnatural physical position, is uncomfortable and may lead to TMJ disorder. Therefore, appliances that pull the jaw forward as a method for addressing snoring have undesirable side effects. Keeping the mouth open without pulling the jaw forward is a more natural approach to addressing snoring since the mouth is opened and closed during all types of normal activities such as talking and eating.

In order to overcome the disadvantages of these and other designs, there is a need for a snore reducing or preventing appliance that keeps the user's mouth open but that prevents the user from breathing through his mouth, unless the nasal passages are blocked by swelling, which may be caused by allergies, colds or the like.

### BRIEF SUMMARY OF THE INVENTION

Accordingly, it is a primary object of the present invention to provide a snore reducing mouthpiece that keeps the user's mouth open at an angle within a certain range that is wide enough to open the airway to the lungs enough to reduce or eliminate snoring, while preventing breathing through the mouth, unless the nasal passages are blocked by swelling, in which case the user instinctively flexes his lips in an attempt to breathe through the closed mouth, which opens the airways and thereby allows the user to breathe through his mouth. If the user remains comfortable during this mouth breathing, it can continue indefinitely. If the user becomes uncomfortable enough to awaken, he can remove the snore reducing mouthpiece and address his nasal issues or simply go back to sleep without using the snore reducing mouthpiece.

This and other objects of the present invention are met by providing a snore reducing mouthpiece that has a unitary body of soft, resilient, tough material that will withstand biting and chewing. The front upper teeth are received and loosely retained in an upper channel portion and the front lower teeth of the user are received and loosely retained in a lower channel portion. The entire snore reducing mouthpiece is held in place by the user's upper and lower lips, gums and teeth, naturally and gently gripping the parts of the mouthpiece that are inserted between the lips from its front side and the gums and teeth from the rear. The front face has no opening of any kind through it, so no breath can pass through it. This prevents the dry mouth that may result from breathing through the mouth.

The snore reducing mouthpiece is entirely inserted into the user's mouth, with the front face of the mouthpiece resting against the inner surface of the user's lips. The mouthpiece is tall enough that it keeps the user's mouth open to or at a certain desired range of angles that keep the mouth open wide enough to reduce or prevent snoring. The mouthpiece is wide enough to block the entire mouth opening when the lips are relaxed, thereby requiring the user to breath through his nose, but the mouthpiece is narrow enough that the user will be able to breathe through his mouth when the lips part. The lips will unconsciously part when breathing through the nose is not possible due to swelling of the nasal passages. In this case, the outer ends of the lips part when the lips are flexed, allowing for sufficient mouth breathing to supply adequate oxygen to the user. This construction solves the problem of a swollen or blocked nasal passages while making nasal breathing the default and usual route for breathing.

The snore reducing mouthpiece also does not grip the teeth tightly, since the teeth fit quite loosely into both the upper channel and the lower channel. This also eliminates the need for custom fitting or custom forming in dentist's office. It also makes this device more comfortable to use than many others that being sold and it can be used while wearing tooth braces, retainers and the like.

Other objects and advantages of the present invention will become apparent from the following description taken in connection with the accompanying drawings, wherein is set forth by way of illustration and example, the preferred embodiment of the present invention and the best mode currently known to the inventor for carrying out his invention.

### BRIEF DESCRIPTION OF THE SEVERAL VIEWS OF THE DRAWING

FIG. 1 is a front view of a snore reducing mouthpiece according to the present invention shown in use by a user.

FIG. 2 is a cross section of the snore reducing mouthpiece of FIG. 1 taken along lines 2-2 of FIG. 1.

FIG. 3 is right-front isometric view of the snore reducing mouthpiece of FIG. 1.

FIG. 4 is a right-rear isometric view of the snore reducing mouthpiece of FIG. 1.

FIG. 5 is a rear view of the snore reducing mouthpiece of FIG. 1.

FIG. 6 is a cross-section of the snore reducing mouthpiece of FIG. 1 taken along the line 6-6 of FIG. 5.

FIG. 7 is a rear view of an alternative embodiment of the snore reducing mouthpiece of FIG. 1.

### DETAILED DESCRIPTION OF THE INVENTION

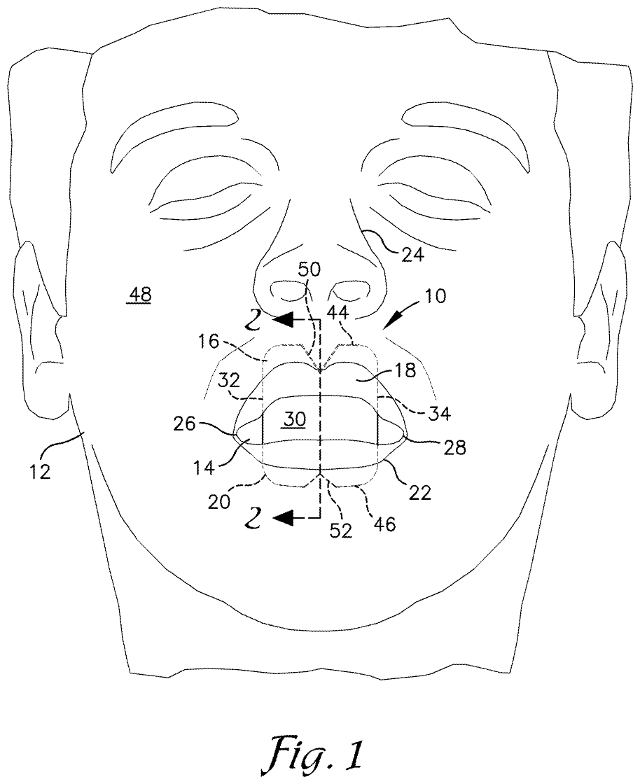

Referring to FIG. 1, a snore reducing mouth piece 10, mouthpiece 10, according to the present invention, is shown being used by a user 12 who places the mouthpiece inside the user's 12 mouth 14 with an upper portion 16 of the mouthpiece 10 lying behind the user's 12 upper lip 18 and a lower portion 20 of the mouthpiece 10 lying behind the user's 12 lower lip 22. The mouthpiece 10 prevents the user 12 from breathing through his mouth when the user's 12 lips 18, 22 are relaxed, requiring the user 12 to breathe though his nose 24, which prevents snoring. When, however the user's 12 nose 24 cannot be used for breathing due to internal swelling or the like, the outer edges of the user's 12 lips 18, 22 open and allow the user 12 to breathe through his mouth 14. In greater detail the right-most ends 26 of the user's 12 upper and lower lips 18, 22 and the left-most ends 28 of the user's 12 upper and lower lips 18, 22 involuntarily open when the user 12 cannot breathe through his mouth 14, allowing the user 12 to continue breathing despite having most of his mouth 14 obstructed by the mouthpiece 10, which has a solid face 30, that is, there are no holes, passages, or the like that would allow air to flow through the face 30, which has a front surface 33. Left and right or left side and right side are stated with respect to the user 12 with the mouthpiece seated in the his mouth 14. The width of the mouthpiece 10, from the right-side edge 32 to the left-side edge 34, must always be less than the width of the user's 12 mouth 14 so that the mouthpiece 10 can never prevent mouth breathing altogether. When the user 12 breathes entirely through his mouth 14, the right left ends 26, 28 of the user's 12 lips 18, 22 open, allowing mouth breathing, and the mouthpiece 10 reduces snoring. The mouthpiece 10 must also be large enough that it cannot be swallowed. All surfaces on the mouthpiece 10 are smooth in order to be more comfortable to the user 12. The mouthpiece 10 is made as a single body 31 from a relatively soft, pliable material that is comfortable to the user 12 and that the user 12 can bite on, clamp between the teeth and not cut or deform it. The mouthpiece 10 is preferably made as a solid body 31, which may be made by molding, machining or any other suitable process. It may also be a hollow body 31, provided that the walls are strong enough to withstand vigorous use.

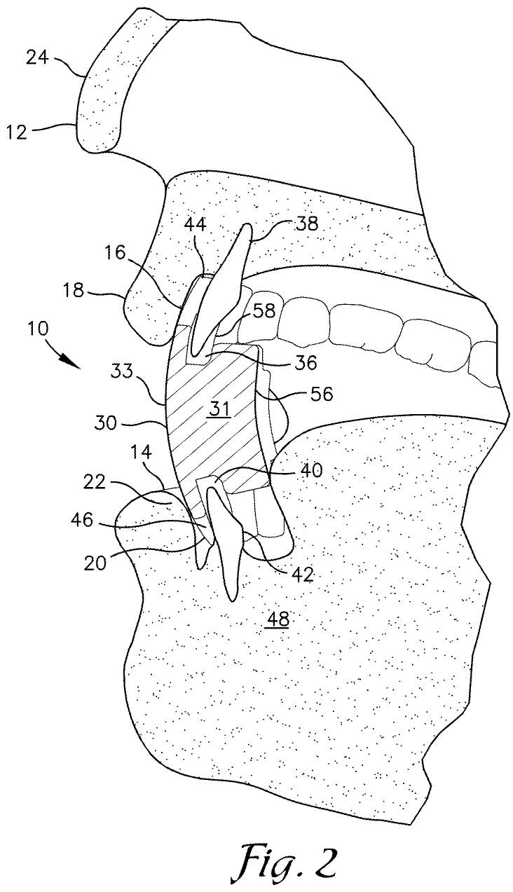

Referring to FIG. 2, the mouthpiece 10 includes an upper channel 36 for receiving and loosely retaining at least some of the front upper teeth 38 of the user 12 and a lower channel 40 for receiving and loosely retaining at least some of the lower front teeth 42 of the user 12. Having the front upper teeth 38 seated in the upper channel 36 and the lower front teeth 42 seated in the lower channel 40 retains the mouthpiece firmly in position for use and prevents it from falling out of the user's 12 mouth 14. The face 30 of the mouthpiece 10 has a uniform outwardly convex curve from the top edge 44 of the face 30 to the bottom edge 46 of the face 30, allowing top edge 44 to be seated easily between the inner surface of the user's 12 upper lip 18 and the user's front upper teeth 38 and allowing the bottom edge 46 of the face 30 to be seated easily between the inside surface of the user's 12 lower lip 22 and the user's 12 front lower teeth 42 and then to maintain a natural and comfortable angle for both the upper and lower lips 18, 22 relative to the user's 12 face 48.

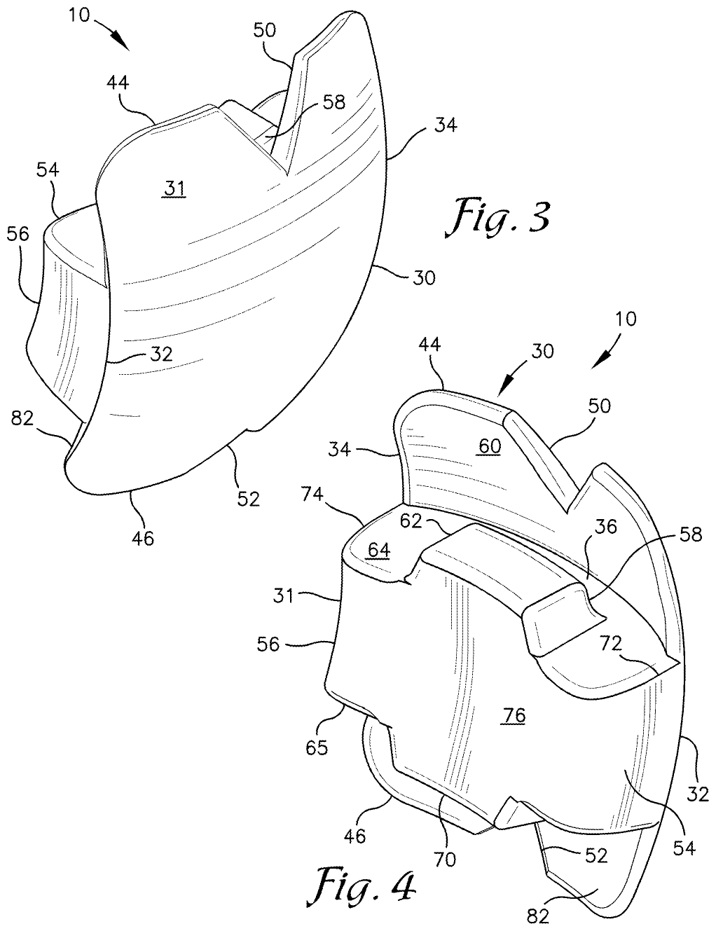

Referring to FIG. 3, the face 30 of the mouthpiece 10 is also uniformly outwardly convex from the right-side edge 32 of the mouthpiece 10 to the left-side edge 34 of the mouthpiece 10, mimicking the left-to-right curvature of the user's face 48 from the vertical centerline of the user's face 48, which increases user 12 comfort. An upper V-shaped notch 50, which is wider at the top than at the bottom, is formed in the center of the edge 44 and receives the maxillary labial frenum of the user 12, while a similar but inverted lower V-shaped notch 52 is formed in the center of the bottom edge 46 of the face 30, with the widest portion of the V-shape along the bottom edge 46 and receives the lower frenum. Both of the notches 50, 52 increase the comfort of the user 12 considerably by receiving the upper and lower frenum, respectively. The upper V-shaped notch 50 has a deeper V-shape than the lower and inverted V-shaped notch 52 does. This structure increases user 12 comfort because the vertical distance of the teeth and gums that is covered by the upper lip is greater than the vertical distance of the teeth and gums that is covered by the lower lip and the upper frenum is larger than the lower frenum. Similarly, for the same reasons, the upper portion of the face 30, that is, the portion of the face 30 that is above the top surface 65 of the bolster portion 56 is greater than the lower portion of the face 30, that is the portion of the face 30 that is below the bottom surface 65 of the bolster portion 56. The right-side portion 54 of a reinforcing bolster portion 56 increases the strength of the mouthpiece 10. An upper rear wall 58, which is part of the reinforcing bolster portion 56, forms the rear wall of the upper channel 36 of the mouthpiece 10.

Referring to FIG. 4, the reinforcing bolster portion 56 extends across the full width of the upper rear surface 60 of the face 30 while an upstanding rear channel wall bolster portion 62 is centered along the width of the mouthpiece 10 and raised above the top surface 64 of the bolster portion 56, forming the upper channel 36 between the rear channel wall bolster 62 and the rear surface 60 of the upper portion of the front face 30, forming an upper front channel wall. The rear channel wall bolster portion 62 runs along only a portion of the width of the mouthpiece 10. A depending rear channel wall bolster 70 has the same dimensions and placement relative to the width of the mouthpiece 10 as the upstanding rear channel wall bolster 62. The upper channel 36 and the lower channel 40 and the portions or members that form them are symmetrical and of the same dimensions in all respects except that the walls forming the upper channel 36 project upwardly and the walls that form the lower channel 40 project downwardly. The right side 54 and the left side 74 of the bolster portion 56 are tapered inwardly from the face 30 to the rear wall 76 of the bolster 56 to so that the sides 72, 74 are drawn away from the user's 12 cheeks, increasing the user's 12 comfort.

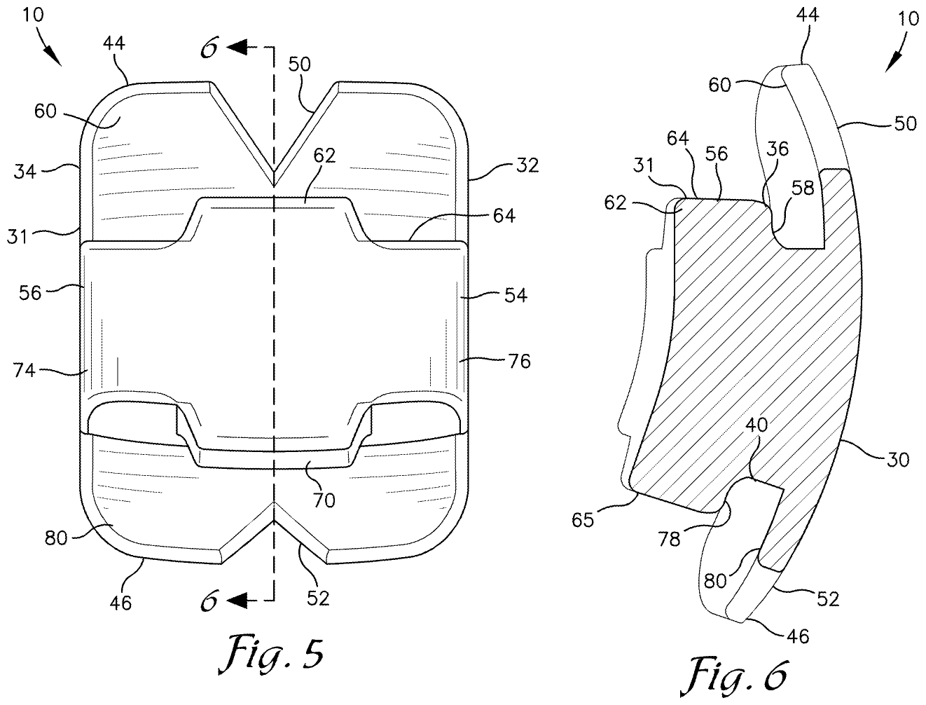

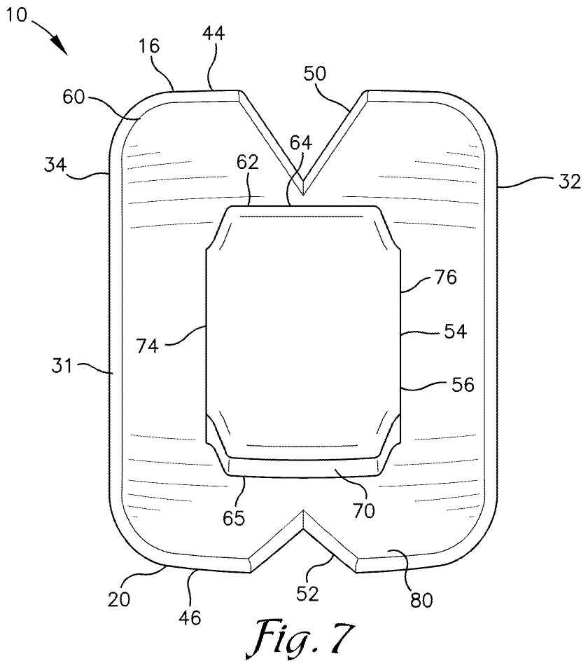

Referring to FIGS. 5, 6, the depending rear channel wall bolster 70 has a forward wall 78 set rearwardly of the lower portion 80 of the lower rear surface 82 of the face 30 of the mouthpiece 10 that forms the lower channel 40. Both the upper and lower channels 36, 40 are wider (from front to back) than the teeth are thick, so that all teeth seated in the channels 36, 40 are loosely seated. This arrangement prevents the mouthpiece from acting like a retainer and so the mouthpiece 10 cannot urge or force the teeth into a different alignment.

Referring to FIG. 7, in an alternative embodiment of the mouthpiece 10, the width of the bolster portion 56 is the same as the width of the upper and lower channels 36, 40, which remain unchanged from the embodiment of FIGS. 5, 6. These portions remain centered in the rear face 60, from side-to-side. This construction saves materials and reduces weight, but does not provide as much support for the upper and lower front teeth 38, 42, which rest along the entire width of the top surface 64 and the bottom surface 65 of the bolster portion 56.

While the present invention has been described in accordance with the preferred embodiments thereof, the description is for illustration only and should not be construed as limiting the scope of the invention. Various changes and modifications may be made by those skilled in the art without departing from the spirit and scope of the invention as defined by the following claims.

I claim:

1. A mouthpiece comprising:
a. a body, said body further comprising a front face and a bolster portion behind said front face and connected to said front face;
b. an upper channel portion for receiving and retaining some of the upper front teeth of a user; and
c. a lower channel portion of said body for receiving and retaining some of the lower front teeth of a user; and
d. a width of said front face that is less than the width of a user's mouth.

2. A mouthpiece according to claim 1 further comprising an upper portion of said font face that is seated between a user's upper lip and the user's upper front teeth and gums.

3. A mouthpiece according to claim 1 further comprising a lower portion of said front face that is seated between a user's lower lip and the user's lower front teeth and gums.

4. A mouthpiece according to claim 1 further comprising an upper V-shaped notch formed in a top edge of said front face wherein said V-shaped notch is wider at top of said V-shaped notch that at the bottom of said V-shaped notch.

5. A mouthpiece according to claim 4 further comprising a lower V-shaped notch formed in a lower edge of said front face wherein said lower V-shaped notch is wider at a bottom of said lower V-shaped notch than at a top of said V-shaped notch.

6. A mouthpiece according to claim 5 wherein said upper V-shaped notch and said lower V-shaped notch are both formed into the center of said width of said front face.

7. A mouthpiece according to claim 1 further comprising an upper channel portion and a lower channel portion that receive and loosely retain at least some of the user's upper front teeth and at least some of the user's lower front teeth, respectively.

8. A mouthpiece according to claim 1 further comprising an upper channel wall bolster portion on said bolster portion, forming an upper rear channel wall and a rear surface of said upper portion of said front face both combing to form said upper front channel wall.

9. A mouthpiece according to claim 8 wherein the width of a top surface of said bolster portion is wider than the width of said upper channel wall bolster portion.

10. A mouthpiece according to claim 1 further comprising a lower channel wall bolster portion on said bolster portion, forming a lower rear channel wall and a lower rear surface of said front face combining to form said lower channel.

11. A mouthpiece according to claim 10 wherein the width of a top surface of said bolster portion is wider than the width of said lower channel wall bolster portion.

12. A mouthpiece according to claim 1 further comprising a tapering of the width of said bolster portion from a front of said bolster portion to a rear wall of said bolster portion.

13. A mouthpiece comprising:
a. a body, said body further comprising a front face and a bolster portion behind said front face and connected to said front face, said front face further comprising an upper portion that is seated between a user's upper lip and the user's upper front teeth and gums and a lower portion of said front face that is seated between a user's lower lip and the user's lower front teeth and gums;
b. an upper channel portion for receiving and retaining some of the upper front teeth of a user; and
c. a lower channel portion of said body for receiving and retaining some of the lower front teeth of a user;
d. an upper V-shaped notch formed in a top edge of said front face wherein said V-shaped notch is wider at top of said V-shaped notch that at the bottom of said V-shaped notch; and
e. a lower V-shaped notch formed in a lower edge of said front face wherein said lower V-shaped notch is wider at a bottom of said lower V-shaped notch than at a top of said V-shaped notch.

14. A mouthpiece according to claim 13 wherein said upper V-shaped notch and said lower V-shaped notch are both formed into the center of said width of said front face.

15. A mouthpiece according to claim 13 further comprising a solid body.

16. A mouthpiece according to claim 13 further comprising front face that is curved convexly outwardly from a top edge of said front face to a bottom edge of said front face and from a left side to a right side of said front face.

17. A mouthpiece according to claim 13 further comprising a tapering of the width of said bolster portion from a front of said bolster portion to a rear wall of said bolster portion.

18. A mouthpiece comprising:
a. a body, said body further comprising a front face and a bolster portion behind said front face and connected to said front face, said front face further comprising an upper portion is seated between a user's upper lip and the user's upper front teeth and gums and a lower portion of said front face that is seated between a user's lower lip and the user's lower front teeth and gums;
b. an upper channel portion for receiving and retaining some of the upper front teeth of a user; and
c. a lower channel portion of said body for receiving and retaining some of the lower front teeth of a user;
d. an upper V-shaped notch formed in a middle of a top edge of said front face wherein said V-shaped notch is wider at top of said V-shaped notch that at the bottom of said V-shaped notch; and
e. a lower V-shaped notch formed in a middle of a lower edge of said front face wherein said lower V-shaped notch is wider at a bottom of said lower V-shaped notch than at a top of said V-shaped notch; and
f. a tapering of the width of said bolster portion from a front of said bolster portion to a rear wall of said bolster portion.

19. A mouthpiece according to claim 18 further comprising front face that is curved convexly outwardly from a top edge of said front face to a bottom edge of said front face and from a left side to a right side of said front face.

20. A mouthpiece according to claim 18 wherein said body further comprises a body that is wide enough to block mouth breathing but narrow enough to allow a user to breath when the user flexes his lips.
